(19) United States
(12) Patent Application Publication
(10) Pub. No.: US 2019/0274620 A1
(43) Pub. Date: Sep. 12, 2019

(54) METHOD FOR DIAGNOSING SCOLIOSIS USING SPATIAL COORDINATES OF BODY SHAPE AND COMPUTER PROGRAM THEREFOR

(71) Applicant: DRIOM CO., LTD., Incheon (KR)

(72) Inventor: Woo Jin CHOI, Incheon (KR)

(73) Assignee: DRIOM CO., LTD., Incheon (KR)

(21) Appl. No.: 15/916,656

(22) Filed: Mar. 9, 2018

(30) Foreign Application Priority Data
Mar. 6, 2018 (KR) 10-2018-0026306

(51) Int. Cl. A61B 5/00; A61B 5/107

(52) U.S. Cl. CPC A61B 5/4566; A61B 5/1079; A61B 5/4576; G06T 7/0012; G06T 2207/30012; G06T 2207/10136; G06T 2200/04; G06T 2207/10016

(57) ABSTRACT

The present invention provided a method for diagnosing scoliosis degree using spatial coordinates may include obtaining spatial coordinates corresponding to left, right and center points of shoulders, pelvis and legs of a subject, calculating both height deviations between left and right shoulders and between left and right pelvis, respectively, obtaining a first scoliosis degree by combining the height deviations, calculating a length deviation between the left and right legs, obtaining a second scoliosis degree by the length deviation and, determining a final scoliosis degree by summing the first and second scoliosis degrees.

### TECHNICAL FIELD

The present invention relates to a method for diagnosing the degree of scoliosis of a subject using spatial coordinates of a few skeletons of a subject, and more particularly, to a method of automatically and easily diagnosing the degree of scoliosis by combining the height deviation between the left and right shoulders, the height deviation between the left and right pelvis and the length deviation between the left and right legs, and a computer program therefor.

### BACKGROUND ART

X-ray radiography and CT scan commonly used as scoliosis diagnostic methods are concerned about the harmful X-ray exposure to the human body, and the universal scans, such as visual measurements and scoliometer, are concerned about the reliability of the results.

And, a diagnostic method based on posture analysis is also used. However, this method requires expensive equipment. In particular, when a gyroscope is used, it is cumbersome since markers must be attached to body, takes a long time to diagnose, and the measured data is not adequately utilized as scoliosis clinical results because of the frequency of errors.

In the case of using infrared ray, which is another diagnostic method of non-radiography scoliosis, marker must be attached to the body. In case of using moire method, there is an inconvenience that his shirt should be taken off. Therefore, another technique for diagnosing scoliosis using cameras is being utilized as an alternative.

Scoliosis diagnosis is one of diseases that can be finally judged by a doctor by analyzing various factors comprehensively. Except for the expensive diagnostic equipment generally used in the hospital, there is no publicly available equipment and algorithms to easily determine the degree of scoliosis in the public.

Meanwhile, technology to diagnose various diseases by recognizing the spatial coordinates of the body of the subject using cameras has been developed. However, it is not enough to be used as judging the diagnosis for scoliosis because the spatial coordinates of the body of the subject are obtained with the space coordinates necessary for judging scoliosis, and analyzed comprehensively.

In addition, for the algorithm used in the equipment for diagnosing scoliosis using cameras, its reliability of judging scoliosis is insufficient because it is diagnosed uniformly by spatial coordinates.

Therefore, the method of utilizing data of some parts of the body skeleton for diagnosing scoliosis and digitizing the degree of abnormality of scoliosis will be very effective, because it could improve its accuracy, the subject can easily recognize the abnormality and decide whether to treat his scoliosis promptly.

It is necessary to develop a method and system that can diagnose the degree of scoliosis inexpensively, promptly and accurately when synthesizing the above contents.

### DISCLOSURE OF THE INVENTION

### Problem to be Solved

Accordingly, the present invention has been made to solve the above-mentioned problems described in the prior art, and it is an object of the present invention to provide a method of diagnosing an abnormality of a scoliosis using a universal non-contact optical analysis apparatus and a computer program therefor.

It is another object of the present invention to provide a method for digitizing the degree of the scoliosis of the subject to easily recognize the state of the scoliosis of the subject, and a computer program therefor.

In addition, it is an another object to maintain the reliability of the test results and to realize the accuracy and reproducibility of the diagnosis, while lowering the anxiety of the radiographic examination.

### Technical Solution

According to an aspect of the present invention, there is provided a method for diagnosing scoliosis degree using spatial coordinates may include obtaining spatial coordinates corresponding to left, right and center points of shoulders, pelvis and legs of a subject, calculating both height deviations between left and right shoulders and between left and right pelvis, respectively, obtaining a first scoliosis degree by combining the height deviations, calculating a length deviation between the left and right legs, obtaining a second scoliosis degree by the length deviation and, determining a final scoliosis degree by summing the first and second scoliosis degrees.

In one preferred form of the invention, summing may be performed by assigning weights to the first and second scoliosis degrees, respectively, and summing up.

The weight of the second scoliosis degree is higher than weight of the second scoliosis degree.

The weight of the second scoliosis degree may be set to 70% and the weight of the first scoliosis degree be set to 30%.

In another preferred form of the invention, it may further comprise calculating whether the height deviation between the left and right shoulders is on the same side or opposite side to the height deviation of the pelvis, before the step of obtaining a second scoliosis degree.

The height deviation between right and left shoulders is divided into 3 mm and 10 mm, the height deviation between right and left pelvis is divided into 3 mm, 6 mm, 10 mm and 15 mm, and the scoliosis degrees are digitized, respectively.

The length deviation between right and left legs is divided into 5 mm, 10 mm, 15 mm, 20 mm, 25 mm and 30 mm, and the scoliosis degrees are digitized, respectively.

According to still another aspect of the present invention, it may be implemented as a computer program stored in a computer-readable recording medium for executing each step of the for diagnosing scoliosis degree using spatial coordinates described above in a computer.

### Advantageous Effects

According to the present invention, it is possible to diagnose even a general-purpose equipment such as Microsoft's keynote, which can greatly reduce the cost and can be used in combination with existing equipment.

In addition, if the present invention is implemented in a smart device such as a smart phone by implementing a diagnosis method of an abnormality on the spine side only, the diagnostic service can be easily used more than the spine side of the subject through a smart device.

Further, according to the present invention, if only the body skeleton coordinate value of the subject can be received even at a remote site, diagnosis can be performed only at the spine side of the subject.

In addition, according to the present invention, various information such as a corrective motion, a correct posture, and a treatment plan according to a diagnosis result of only the spinal column side of the subject can be provided.

### DESCRIPTION OF DRAWINGS

FIG. 1 is a diagram for describing a system network configuration of a diagnosis system for diagnosing scoliosis abnormality using spatial coordinate analysis of a body.

FIG. 2 is a flow chart showing a process of diagnosing scoliosis degree using spatial coordinates according to an embodiment of the present invention.

FIG. 3 is a table for explaining the first reference value according to an embodiment of the present invention.

FIG. 4 is a table for explaining the second reference value according to an embodiment of the present invention.

FIG. 5 is a table for explaining the final scoliosis abnormality according to an embodiment of the present invention.

FIG. 6 is acquire spatial coordinates corresponding to body of subject from sensor device according to an embodiment of the present invention.

FIG. 7 is a flow chart showing a process of diagnosing scoliosis degree using spatial coordinates according to an another embodiment of the present invention.

FIG. 8 is a table for explaining the first reference value according to another embodiment of the present invention.

### MODE FOR INVENTION

The present invention may have various modifications and shapes, but will be explained as an example referring to figures in detail.

However, the specification and cases below are for showing embodiments of the present invention but only for examples. the present invention may, however, be embodied in many different forms and should not be construed as limited to the example embodiments set forth herein.

It will be understood that, although the terms first, second, third etc. may be used herein to describe various elements, components, regions, layers and/or sections, these elements, components, and/or sections should not be limited by these terms. These terms are only used to distinguish one element, component, region, layer or section from another region, layer or section. Thus, a first element, component, or section discussed below could be termed a second element, component, or section without departing from the teachings of the present invention.

The terminology used herein is for the purpose of describing particular example embodiments only and is not intended to be limiting of the present invention. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this invention belongs.

It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Hereinafter, exemplary embodiments of the present invention will be described with reference to the accompanying drawings.

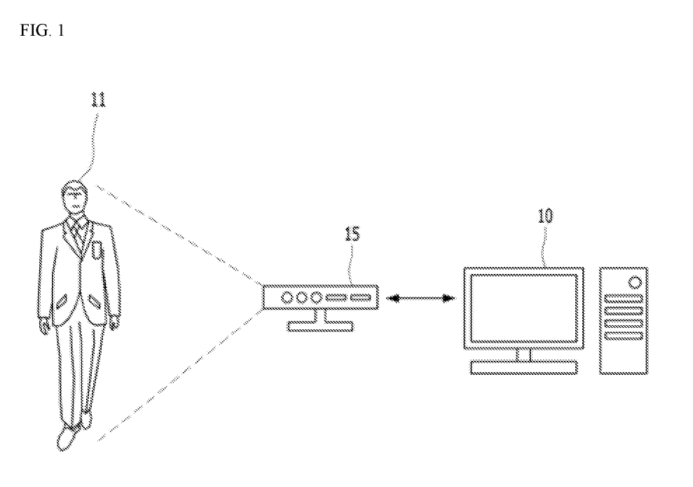

FIG. 1 is a diagram for describing a system network configuration of a diagnosis system for diagnosing scoliosis abnormality using spatial coordinate analysis of a body.

Referring to FIG. 1, a system for diagnosing scoliosis abnormality using spatial coordinates includes a sensor device 15 acquiring spatial coordinates data corresponding to skeleton portion of a subject 11, and an analyzer 10 for receiving the data from the sensor device 15 and analyzing the data to diagnose the scoliosis abnormality of the subject 11.

The sensor device 15 has a sensor and/or cameras to acquire the spatial coordinates of a plurality of reference points on skeleton portion of a subject 11. The sensor device 15 may be preferable to use an apparatus using non radiation such as IR (Infrared) or TOF (Time Of Flight).

A typical sensor device 15 is Kinect from Microsoft. Kinect 1.0 is an IR optical system and Kinect 2.0 is a TOF system, which both systems incorporate skeleton tracking system being capable of recognizing about 20 skeleton parts.

The analyzer 10 for automatically analyzing scoliosis abnormality by software includes a communication unit for processing communication with the sensor device 15, a database for storing data and a diagnostic processing unit. It may further include a user terminal (not shown) for providing a diagnosis result and a device configured to be provided with various information such as correction exercise according to the diagnosis result, right postures and/or medical treatment plan.

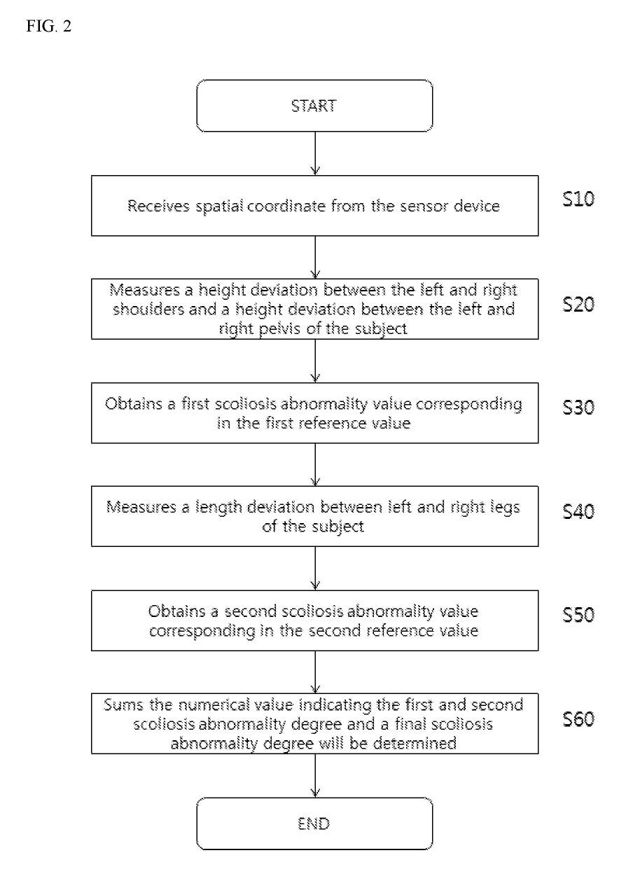

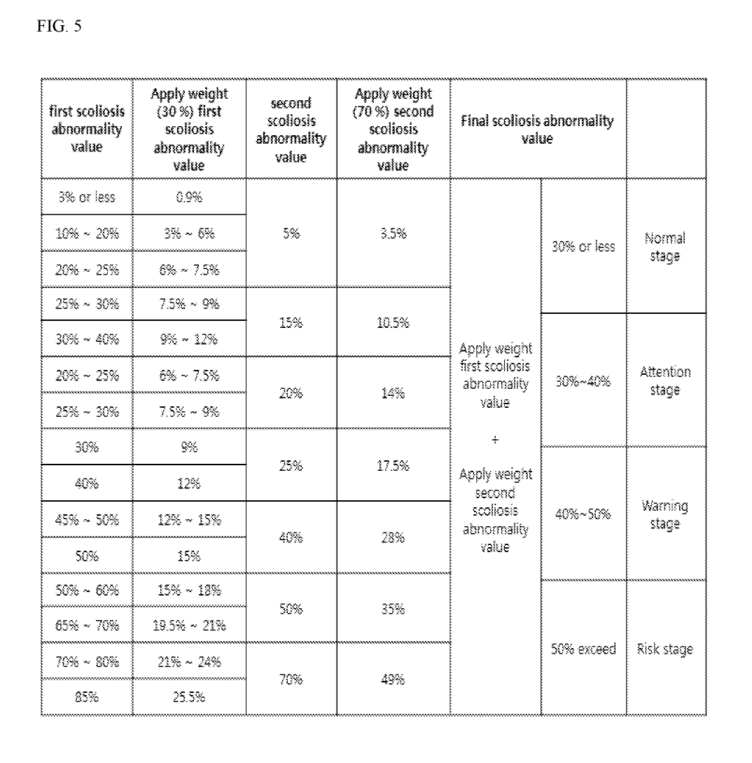

On the other hand, the diagnostic system for diagnosing scoliosis abnormality using spatial coordinates according to the present invention may be performed by a smart device such as smart phone, tablet, and PDA. For example, spatial coordinates of a plurality of reference points of skeleton portions of a subject may be acquired through cameras and/or sensors provided in the smartphone, and diagnosing of scoliosis abnormality be performed through an application installed in the smartphone. Therefore, the present invention may be applied to any device capable of acquiring spatial coordinates of skeletons of a subject. Hereinafter, a method for diagnosing scoliosis degree using spatial coordinates according to an embodiment of the present invention will be described with reference to FIGS. 2 to 6.

The analyzer 10 receives spatial coordinate from the sensor device 15 on multiple reference points of body of subject. (S10).

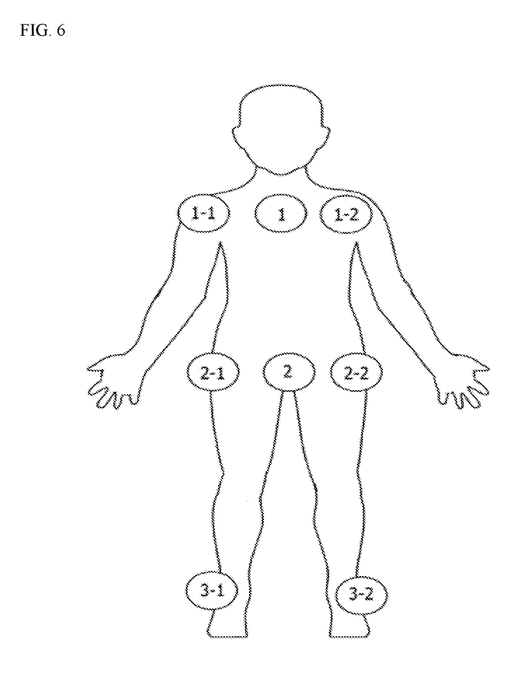

The commonly available sensor device 15 can acquire spatial coordinates corresponding to eyes, shoulders, a midpoint between the shoulders, elbows, wrists, pelvis, a midpoint between the pelvis, knees, ankles, thumb toes. It is effective to utilize only the minimum spatial coordinates in order to accurately diagnose scoliosis abnormality. That is, in order to diagnose the scoliosis abnormality, it is preferable to exclude spatial coordinates corresponding to other skeletons than the left and right shoulders, pelvis and legs because it may cause confusion in the analysis. Therefore, according to the embodiment of the present invention, as shown in FIG. 6, spatial coordinates corresponding to reference points of shoulders (1-1, 1-2), shoulder midpoint point (1) between the reference points of shoulders, reference points of pelvis (2-1, 2-2), pelvis midpoint point (2) between the reference points of pelvis and ankle reference points (3-1, 3-2) are utilized. At this time, the shoulder midpoint point (1) and pelvis midpoint point (2) may be recognized by the sensor device 15 or be calculated by the analyzer 10 with the spatial coordinates corresponding to the reference points of shoulders (1-1, 1-2) and reference points of pelvis (2-1, 2-2) received from the sensor device 15.

By using only the minimum spatial coordinate data necessary for diagnosing the abnormality of a subject's scoliosis, it is possible to prevent a situation in which it is difficult to recognize spatial coordinates necessary for diagnosing the abnormality of a subject's scoliosis due to overlapping with spatial coordinates of other skeletons. In addition, it is possible to diagnose the abnormality of a subject's scoliosis accurately, and the recognition time can be shortened by utilizing only the spatial coordinates of the minimal skeletal region.

The analyzer 10 measures a height deviation between the left and right shoulders and a height deviation between the left and right pelvis of the subject, respectively. (S20).

The height deviation between the left and right shoulders is determined by the vertically spaced distance between the reference points of shoulders (1-1, 1-2). When a subject's feet is on the xy plane and his upper side is on z axis, the reference point coordinates are represented by three-dimensional spatial coordinates (x, y, z) and the height deviation between the left and right shoulders can be calculated by the difference between the z value of the reference point (1-1) and the z value of the reference point (1-2).

Likewise, the height deviation between the left and right pelvis can be calculated by the difference between the z value of the reference point (2-1) and the z value of the reference point (2-2).

The analyzer 10 combines the height deviation between the left and right shoulders and the height deviation between the left and right pelvis analyzed as described above. A first scoliosis abnormality value is obtained by corresponding to the combination within the first reference value. (S30).

The first reference value is previously obtained by dividing the height deviation between the left and right shoulders and the height deviation between the left and right pelvis into a plurality of sections by correlating the diagnostic results corresponding to each combination of the sections. The first reference value can be newly input and corrected by the administrator or can be newly constructed through the statistics based on the past diagnosis history.

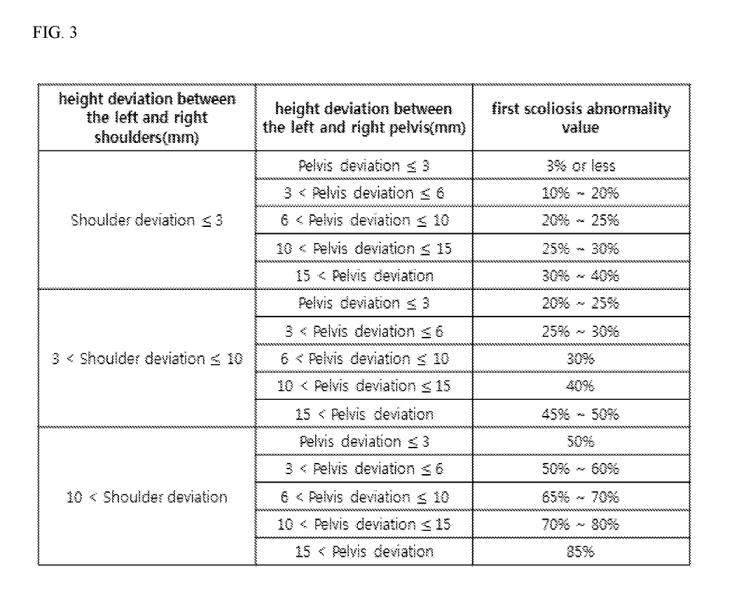

As shown in FIG. 3, the first reference value is defined as a difference in height deviation between the right and left shoulders of 3 mm or less, 3 mm to 10 mm, and 10 mm or more, and the height deviation of the right and left pelvises is 3 mm or less, between 3 mm and 6 mm, between 6 mm and 10 mm and exceeding 15 mm, and scoliosis abnormality degree corresponding to the tested results will be a first scoliosis abnormality degree. At this time, the numerical value is displayed as a percentage, so that the subject can easily recognize scoliosis abnormality degree.

For example, when a height deviation between left and right shoulders of a subject is 5 mm and the height deviation between the left and right pelvises is 7 mm, the height deviation between left and right shoulders corresponds to the interval between 3 mm and 10 mm, and the height deviation between left and right pelvises is 6 mm and 10 mm. Therefore, the first scoliosis abnormality degree will be matched as 30%, which corresponds to the combination of both the height deviation of 3 mm and 10 mm between left and right shoulders and the height deviation of 6 mm and 10 mm between right and left pelvises.

The analyzer 10 measures a length deviation between left and right legs of the subject. (S40).

The length deviation between left and right legs may be calculated by the vertically spaced distance between the reference points of pelvises (2-1, 2-2) and the reference points of ankles (3-1, 3-2).

The analyzer 10 obtains, as above described, a second scoliosis abnormality value corresponding in the second reference value. (S50).

The second reference value is previously obtained by dividing the length deviation between left and right legs into a plurality of sections by correlating the diagnostic results corresponding to the sections. The second reference value can be newly input and corrected by the administrator or can be newly constructed through the statistics based on the past diagnosis history.

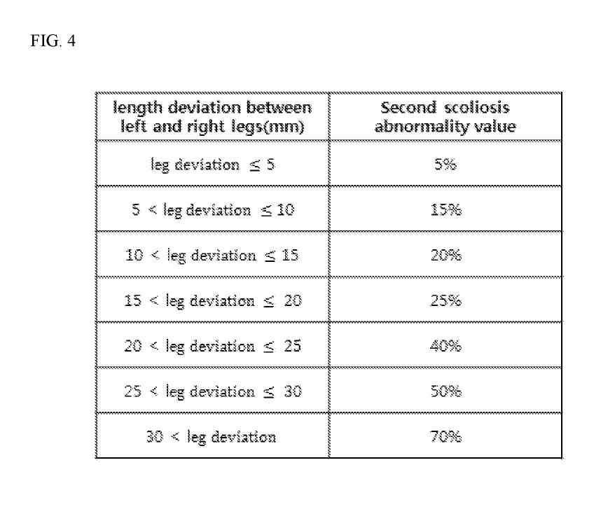

As shown in FIG. 4, the second reference value is defined as a difference in height deviation between right and left legs of 5 mm or less, 5 mm to 10 mm, 10 mm or 15 mm, 15 mm or 20 mm, 20 mm or 25 mm, 25 mm or 30 mm, and 30 mm or more, and scoliosis abnormality degree corresponding to the tested results will be a second scoliosis abnormality degree. At this time, the numerical value is displayed as a percentage, so that the subject can easily recognize scoliosis abnormality degree.

For example, when a leg deviation between left and right legs of a subject is 6 mm, the leg deviation between left and right legs corresponds to the interval between 5 mm and 10 mm. Therefore, the second scoliosis abnormality degree will be matched as 15% which corresponds to the height deviation of 5 mm and 10 mm.

The analyzer 10 sums the numerical value indicating the first and second scoliosis abnormality degree and a final scoliosis abnormality degree will be determined. (S60).

The final scoliosis abnormality degree may be calculated by giving the weights of the first and second scoliosis reference values, respectively in the process of summing, thereby making accuracy of the final scoliosis abnormality degree. Scoliosis differs from person to person, but it is more affected by leg length deviation than shoulder height deviation. And, through various experiments, it has been found that it is accurate to determine the final scoliosis abnormality degree by giving a weight higher than the first reference value to the second reference value. More preferably, a weight value of 70% is given to the second reference value, and a weight value of 30% is given to the first reference value.

For example, let's assume that a height deviation between left and right shoulders of a subject is 5 mm and the height deviation between the left and right pelvises is 7 mm, the height deviation between left and right legs is 6 mm. The first and second scoliosis abnormality degree will be matched as 30% and 15%, respectively. At this time, if the weights are given to the first and second scoliosis abnormality degrees 30% and 70%, respectively, as described above, the final scoliosis abnormality degree is determined to 19.5% by the equation of 30%*0.3+15%*0.7.

The analyzer 10 may classify normal stage in case of the degree of the final scoliosis abnormality degree is less than 30%, attention stage in case of 30% and 40% interval, in the warning stage in the case of the 40% and 50% and risk stage in the case of exceeding 50%, and provide the result information to the subject through various methods such as alarm, color conversion, highlight, and display.

For example, in the case of a subject's height deviation between left and right shoulders of a subject is 5 mm and the height deviation between the left and right pelvises is 7 mm and the height deviation between left and right legs is 6 mm, the final scoliosis abnormality degree will be matched as 19.5%, which corresponds to the normal stage.

The above stages such as normal, attention, warning and risk are classified by less than 30%, between 30% and 40%, between 40% and 50% and exceeding 50%, those can be newly input and corrected by the administrator or be newly constructed through the statistics based on the past diagnosis history.

In the above example, the first scoliosis abnormality degree is obtained and the second scoliosis abnormality degree is obtained. However, after obtaining the second scoliosis abnormality degree, the first second scoliosis abnormality degree may be obtained.

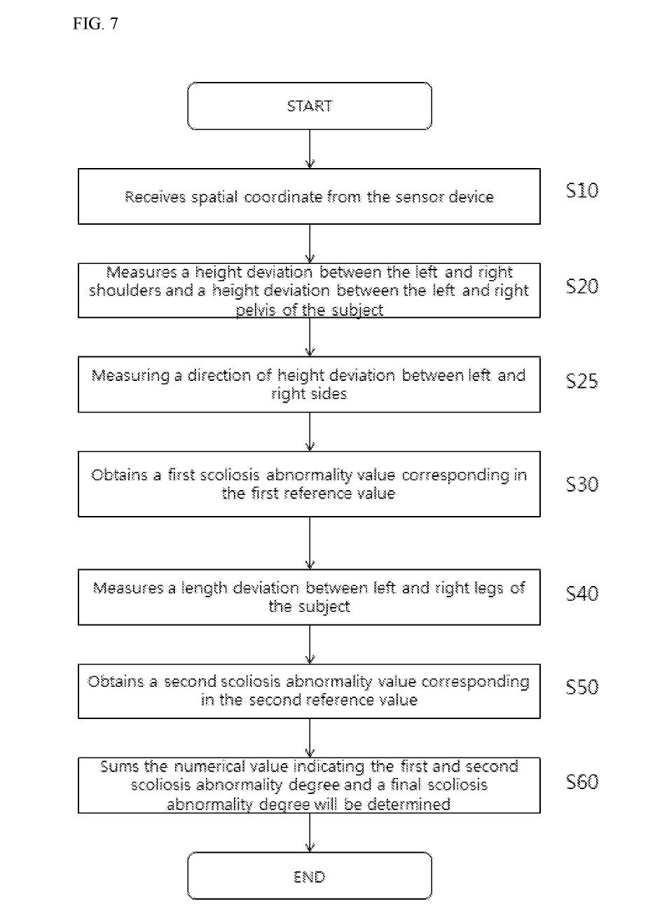

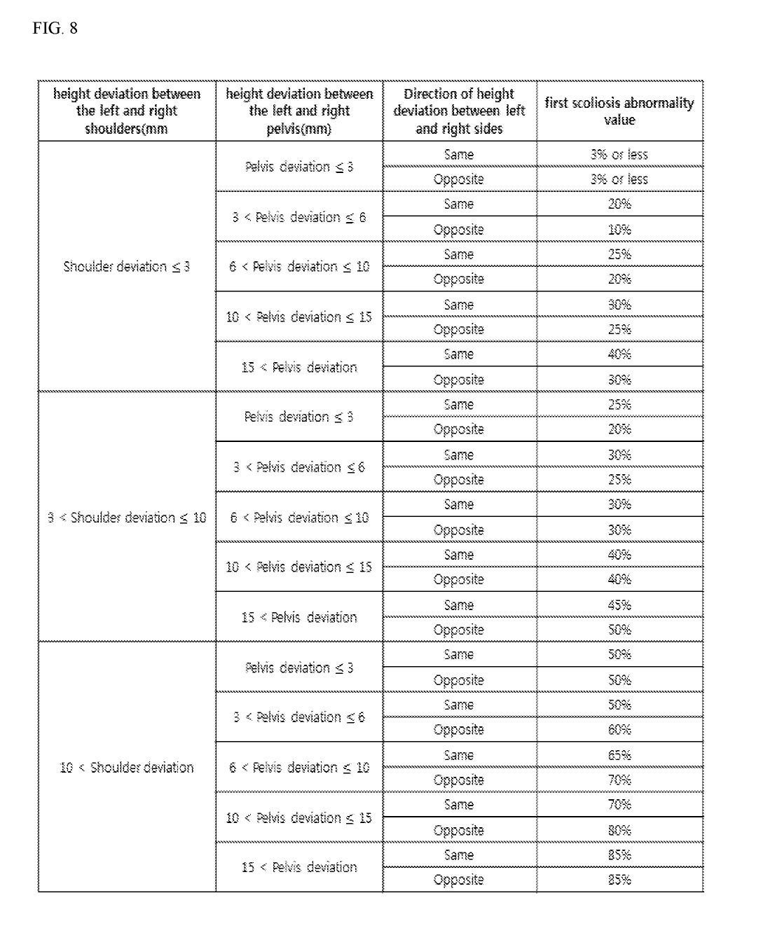

A method for diagnosing scoliosis degree using spatial coordinates according to another embodiment of the present invention will be described with reference to FIG. 7. FIG. 8 a table showing a first reference value indicating scoliosis degree based on height deviations between the left and right shoulders and pelvises and, direction of height deviation between left and right sides.

A method for diagnosing scoliosis abnormality degree using spatial coordinates according to another embodiment of the present invention, as described in FIG. 7, diagnose a first scoliosis abnormality degree by measuring a height deviation between the left and right shoulders and a height deviation between the left and right pelvis of the subject, respectively (S20), and measuring a direction of height deviation between left and right sides. (S25).

Specifically, the method of determining the height deviation direction between right and left sides is to calculate whether the height difference between the shoulder and the pelvis of each of the left and right sides is higher or lower than the difference between the shoulder center point (1) and the center of the pelvis, and judges whether it is the same or opposite position. At this time, the same position is defined as the height difference between the shoulder and the pelvis of each of the left and right sides is the same or lower than the height difference between the center points (1 and 2), and is defined as the opposite side when it is higher.

And, a length deviation between left and right legs is measured (S40), and, as above described, a second scoliosis abnormality value corresponding in the second reference value. (S50). Finally, by summing the numerical value indicating the first and second scoliosis abnormality degree, a final scoliosis abnormality degree will be determined. (S60), which this process is the same as the above-described and a detailed description thereof will be omitted.

FIG. 8 is a table for explaining the first reference value according to another embodiment of the present invention, which additionally reflects a direction of the height difference between the shoulder and the pelvis on right and left sides with Table 3.

For example, when a height deviation between left and right shoulders of a subject is 5 mm and the height deviation between the left and right pelvises is 7 mm, the first scoliosis abnormality degree will be matched as 30% in the case of same position.

Methods based on the examples of performing this invention may be implemented in the form of program instructions that can be performed through a variety of computer tools and recorded on a computer readable medium. The computer readable medium may include program instructions, data files, or a combination of data structures. The program instructions recorded on the media above may be those specifically designed and constructed for this invention or may be communicated and available to computer software vendors. The computer readable media includes hardware devices configured specifically to store and perform program commands. Examples of computer-readable media include magnetic media (such as hard disk, floppy disk, and magnetic media), optical media (such as CD-ROM, DVD, and optical media), magneto-optical media (such as floptical disk), ROM, RAM and flash memory. Examples of program instructions include the advanced language code that can be executed by the computer using the computer's interpreter, as well as the system language code, which is created by the compiler.

It will be apparent to those skilled in the art that various modifications and variation can be made in the present invention without departing from the spirit or scope of the invention. Thus, it is intended that the present invention cover the modifications and variations of this invention provided they come within the scope of the appended claims and their equivalents.

What is claimed is:

1. A method for determining of scoliosis degree using spatial coordinates, wherein the method comprises;
obtaining spatial coordinates corresponding to left, right and center points of shoulders, pelvis and legs of a subject;
calculating both height deviations between left and right shoulders and between left and right pelvis, respectively;
obtaining a first scoliosis degree by combining the height deviations;
calculating a length deviation between the left and right legs;
obtaining a second scoliosis degree by the length deviation; and,
determining a final scoliosis degree by summing the first and second scoliosis degrees.

2. The method according to claim 1, wherein the summing is performed by assigning weights to the first and second scoliosis degrees, respectively, and summing up.

3. The method of claim 3, wherein weight of the second scoliosis degree is higher than weight of the second scoliosis degree.

4. The method of claim 3, wherein the weight of the second scoliosis degree is set to 70% and the weight of the first scoliosis degree is set to 30%.

5. The method according to claim 1, further comprises calculating whether the height deviation between the left and right shoulders is on the same side or opposite side to the height deviation of the pelvis, before the step of obtaining a second scoliosis degree.

6. The method according to claim 1, wherein the height deviation between right and left shoulders is divided into 3 mm and 10 mm, the height deviation between right and left pelvis is divided into 3 mm, 6 mm, 10 mm and 15 mm, and the scoliosis degrees are digitized, respectively.

7. The method according to claim 1, wherein the length deviation between right and left legs is divided into 5 mm, 10 mm, 15 mm, 20 mm, 25 mm and 30 mm, and the scoliosis degrees are digitized, respectively.

8. A computer program stored on a medium for carrying out the method of claim 1.
